(19) United States
(12) Patent Application Publication
(10) Pub. No.: US 2023/0149701 A1
(43) Pub. Date: May 18, 2023

(54) Active Expandable Sheath

(71) Applicant: Boston Scientific Scimed Inc., Maple Grove, MN (US)

(72) Inventors: Qian Liu, Plymouth, MN (US); Brian R. Reynolds, Ramsey, MN (US); Timothy Lawrence Rubesch, Blaine, MN (US)

(21) Appl. No.: 17/989,122

(22) Filed: Nov. 17, 2022

Related U.S. Application Data
(60) Provisional application No. 63/280,445, filed on Nov. 17, 2021.

(51) Int. Cl. A61M 60/865; A61M 25/01; A61M 60/226; A61M 60/411; A61M 60/13

(52) U.S. Cl. CPC A61M 60/865; A61M 25/0147; A61M 60/226; A61M 60/411; A61M 60/13

(57) ABSTRACT

An introducer sheath for using in delivering a device includes a proximal end opposite a distal end and a body portion, the body portion defining a lumen of the introducer sheath configured for receiving the device, a first portion of the introducer sheath having a compressed configuration defined by a compressed diameter and an expanded configuration defined by an expanded diameter, and at least one actuator configured for transitioning the first portion from the compressed diameter to the expanded diameter.

### CROSS REFERENCE TO RELATED APPLICATION

This application claims priority to Provisional Application No. 63/280,445, filed Nov. 17, 2021, which is herein incorporated by reference in its entirety.

### TECHNICAL FIELD

The present disclosure relates to the field of introducer sheaths for use in the intravascular delivery of medical devices. More particularly, the present disclosure relates to the use of an actively expandable introducer sheath for the intravascular delivery of percutaneous medical devices.

### BACKGROUND

In various procedures, for example procedures for intravascularly delivering a medical device, for example a percutaneous device, an introducer sheath is introducer into a blood vessel, for example a femoral artery, and the medical device and/or tools may be inserted through the introducer sheath for introduction into the blood vessel. However, for the delivery of certain medical devices, the introducer sheath may need to be formed with a diameter that is approximately equal to or greater than the diameter of the blood vessel. Insertion of the introducer sheath into the blood vessel with a large diameter and maintaining the positioning of the introducer sheath with the large diameter may cause damage to and/or tear the blood vessel or reduce blood flow therethrough.

It is known in the art for an introducer sheath to be inserted into the blood vessel with a compressed diameter and be passively expanded by the medical device as the medical device is delivered through the introducer sheath. However, such passive expansion may require a high force for pushing the medical device through the introducer sheath, which can lead to damaging the medical device and/or the surrounding blood vessel.

### SUMMARY

In Example 1, an introducer sheath for use in delivering a device includes a proximal end opposite a distal end and a body portion, the body portion defining a lumen of the introducer sheath configured for receiving the device, a first portion of the introducer sheath having a compressed configuration defined by a compressed diameter and an expanded configuration defined by an expanded diameter, and at least one actuator configured for transitioning the first portion from the compressed diameter to the expanded diameter.

In Example 2, the introducer sheath of Example 1 includes wherein the actuator includes at least one pull wire.

In Example 3, the in troducer sheath of Example 1, includes wherein at least the first portion of the introducer sheath is composed of a braided wire.

In Example 4, the introducer sheath of Example 2, further includes wherein the at least one pull wire is an extension of the braided wire of the first portion.

In Example 5, the introducer sheath of Example 1, further includes wherein the first portion extends along an entire length of the introducer sheath.

In Example 6, the introducer sheath of Example 1 further includes wherein the first portion extends from the distal end and extends a length that is less than a length of the entire introducer sheath.

In Example 7, the introducer sheath of Example 6 further includes wherein the introducer sheath comprises a second portion that extends from the proximal end and extends a length less than a length of the entire introducer sheath, and wherein the second portion is defined by the expanded configuration and does not compress to the compressed configuration.

In Example 8, the introducer sheath of Example 7 further includes wherein when in the compressed configuration, the first portion is defined by a size of 9 French.

In Example 9, the introducer sheath of Example 7 further includes wherein when in the expanded configuration, the first portion is defined by a size of 16 French.

In Example 10, a method for delivering a percutaneous device into a patient includes inserting an introducer sheath into a vessel of the patient, the introducer sheath comprising a proximal end opposite a distal end, a body portion extending between the proximal end and the distal end and the body portion defining a lumen, and the introducer sheath having a first portion extending from the distal end wherein the first portion has a compressed configuration defined by a compressed diameter and an expanded configuration defined by an expanded diameter, the first portion having the compressed diameter when inserting the introducer sheath and actuating an actuator of the introducer sheath such that the first portion expands from the compressed diameter to the expanded diameter. The method further includes inserting the percutaneous device into the introducer sheath and guiding the percutaneous device to a target position in the patient.

In Example 11, the method of Example 10 further includes wherein the actuator includes at least two pull wires extending from the proximal end of the introducer sheath.

In Example 12, the method of Example 11 further includes wherein the introducer sheath is composed of a braided wire and wherein the at least two pull wires extend from the braided wire.

In Example 13, the method of Example 10 further includes wherein the introducer sheath comprises a second portion extending from the proximal portion having the expanded configuration defined by the expanded diameter.

In Example 14, the method of Example 13 further includes wherein the second portion maintains the expanded configuration.

In Example 15, the method of Example 11 further includes wherein the method further includes the step of compressing the first portion from the expanded configuration to the compressed configuration through releasing tension of the at least two pull wires.

In Example 16, an introducer sheath for use in delivering a percutaneous device includes a proximal end opposite a distal end and a body portion extending between the proximal portion and the distal portion, the body portion defining a lumen of the introducer sheath configured for receiving at least one percutaneous device and at least one actuator that is configured for transitioning the first portion from the compressed diameter to the expanded diameter and configured for actuation prior to insertion of the percutaneous device through the introducer sheath.

In Example 17, the introducer sheath of Example 16 further includes wherein the actuator includes at least one pull wire.

In Example 18, the introducer sheath of Example 16 further includes wherein at least the first portion of the introducer sheath is composed of a braided wire.

In Example 19, the introducer sheath of Example 18 further includes wherein the at least one pull wire is an extension of the braided wire of the first portion.

In Example 20, the introducer sheath of Example 16 further includes wherein the first portion extends along an entire length of the introducer sheath.

In Example 21, the introducer sheath of Example 16 further includes wherein the first portion extends from the distal end of the body portion and extends a length less than an entire length of the introducer sheath.

In Example 22, the introducer sheath of Example 21 further includes wherein the introducer sheath comprises a second portion that extends from the proximal end and extends a length less than an entire length of the introducer sheath, and wherein the second portion is defined by the expanded configuration and does not compress to the compressed configuration.

In Example 23, the introducer sheath of Example 21 further includes wherein when in the compressed configuration, the first portion is defined by a size of 9 French.

In Example 24, the introducer sheath of Example 16 further includes wherein when in the expanded configuration, the first portion is defined by a size of 16 French.

In Example 25, a percutaneous device delivery system for delivering a percutaneous device includes the percutaneous device having an impeller housing for supporting an impeller, a motor configured to rotatably drive the impeller within the impeller housing and an introducer sheath for receiving the percutaneous device, the introducer sheath having a proximal end, a distal end, and a body portion extending between the proximal portion and the distal portion, and the body portion defining a lumen. The introducer sheath further includes a first portion extending from the distal end of the introducer sheath, the first portion having a compressed configuration defined by a compressed diameter and an expanded configuration defined by an expanded diameter and an actuator configured for transitioning the first portion from the compressed configuration to the expanded configuration after actuation.

In Example 26, the system of Example 25 further includes wherein the actuator includes a plurality of pull wires extending from the proximal end of the introducer sheath.

In Example 27, the system of Example 25 further includes wherein at least the first portion is composed of a braided wire.

In Example 28, the system of Example 25 further includes wherein the introducer sheath comprises a second portion extending from the proximal end, and wherein the second portion is defined by the compressed diameter.

In Example 29, the system of Example 28 further includes wherein the second portion does not compress to the compressed configuration.

In Example 30, a method for delivering a percutaneous device into a patient includes inserting an introducer sheath into a vessel of the patient, the introducer sheath comprising a proximal end opposite a distal end, a body portion extending between the proximal end and the distal end and the body portion defining a lumen, and the introducer sheath having a first portion extending from the distal end wherein the first portion has a compressed configuration defined by a compressed diameter and an expanded configuration defined by an expanded diameter, the first portion having the compressed diameter when the inserting the introducer sheath and actuating an actuator of the introducer sheath such that the first portion expands from the compressed diameter to the expanded diameter. The method further includes inserting the percutaneous device into the introducer sheath and guiding the percutaneous device to a target position in the patient.

In Example 31, the method of Example 30 further includes wherein the actuator includes at least two pull wires extending from the proximal end of the introducer sheath.

In Example 32, the method of Example 31 further includes wherein the introducer sheath is composed of a braided wire and wherein the at least two pull wires extend from the braided wire.

In Example 33, the method of Example 30 further includes wherein the introducer sheath comprises a second portion extending from the proximal portion having the expanded configuration defined by the expanded diameter.

In Example 34, the method of Example 33 further includes wherein the second portion maintains the expanded configuration.

In Example 35, the method of Example 31 further includes wherein the method further includes the step of compressing the first portion from the expanded configuration to the compressed configuration through releasing tension of the at least two pull wires.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a side view of an introducer sheath after insertion into a blood vessel in a compressed configuration, in accordance with embodiments of the present disclosure.

FIG. 2 illustrates a side view of an introducer sheath after insertion into a blood vessel in an expanded configuration, in accordance with embodiments of the present disclosure.

FIG. 3 illustrates a side view of an introducer sheath after insertion into a blood vessel in an expanded configuration, in accordance with embodiments of the present disclosure.

FIG. 4 illustrates a sectional side cutaway figure showing the introducer sheath of FIG. 2 during delivery of the percutaneous circulatory support device positioned within the introducer sheath, in accordance with embodiments of the present disclosure.

FIG. 5 is a flow chart illustrating a method of delivering the percutaneous circulatory support device of FIG. 4, in accordance with embodiments of the present disclosure.

### DETAILED DESCRIPTION

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

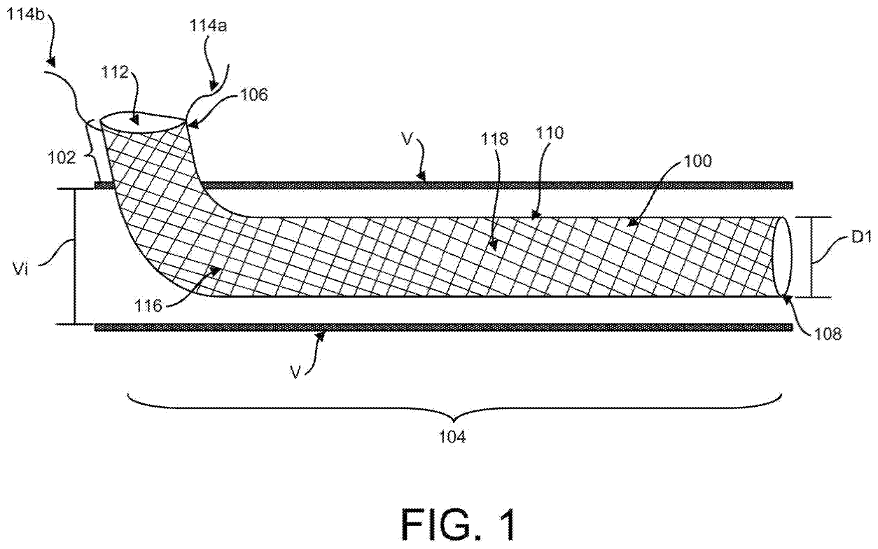

FIG. 1 illustrates a side cross sectional view of a blood vessel V with an introducer sheath 100 inserted at least partially into the blood vessel V. The introducer sheath 100 comprises an outer portion 102 that is positioned outside of the blood vessel V for access by the physician or operator of the introducer sheath 100 and an inner portion 104 that is positioned within the blood vessel V. The introducer sheath 100 includes a proximal end 106 opposite a distal end 108 and a body portion 110 extending between the proximal end 106 and the distal end 108. The body portion 110 defines a lumen 112 of the introducer sheath 100. In the illustrative embodiment of FIG. 1, the lumen 112 is cylindrical, however various other configurations of the lumen 112 may be incorporated.

As illustrated, the introducer sheath 100 is defined by a braided configuration illustrated by braided wires 116, however various other configurations of the introducer sheath 100 may be incorporated as well. The braided wires 116 may define a layer of the introducer sheath 100 composed of a metallic material, for example nitinol or stainless steel. The introducer sheath 100 may additionally comprise an outer covering 118 positioned over the braided wires 116. In embodiments, the outer covering 118 may be formed of an elastomer. This provides the advantage of a smooth outer surface of the introducer sheath 100 to reduce the potential of thrombus while positioned within the blood vessel V. There additionally may be a lubricious coating positioned around the outer covering 118 to increase the ease with which the introducer sheath 100 is inserted. Various other configurations of the introducer sheath 100 may be incorporated and the above examples are provided just as an example.

As illustrated in FIG. 1, the inner portion 104 of the introducer sheath 100 comprises a compressed configuration and an expanded configuration. In embodiments, at least a portion of the introducer sheath 100 is configured for being defined by the compressed configuration in a resting state of the introducer sheath 100. The compressed configuration is defined by a first, compressed diameter D1, as illustrated by the inner portion 104 of the introducer sheath 100. In various embodiments, the outer portion 102 is also defined by the first and compressed diameter Dl. However, in various embodiments, the outer portion 102 may have a different diameter, for example an expanded diameter, as will be discussed further herein. In some embodiments, when in the compressed configuration, the size of the introducer sheath 100 is a value of approximately 9 Fr. In other embodiments, the size of the introducer sheath 100 may be smaller or larger than 9 Fr. As shown in FIG. 1, the diameter D1 is smaller than the inner diameter Vi of the blood vessel V and thus sized to reduce the force that is placed against the blood vessel V while the introducer sheath 100 is inserted into the incision site of the blood vessel V. This configuration of introducer sheath 100 reduces the amount of force exerted against the blood vessel V during insertion of the introducer sheath 100, and may reduce the potential damage to the blood vessel V and the likelihood that the introducer sheath 100 will substantially affect blood flow through blood vessel V.

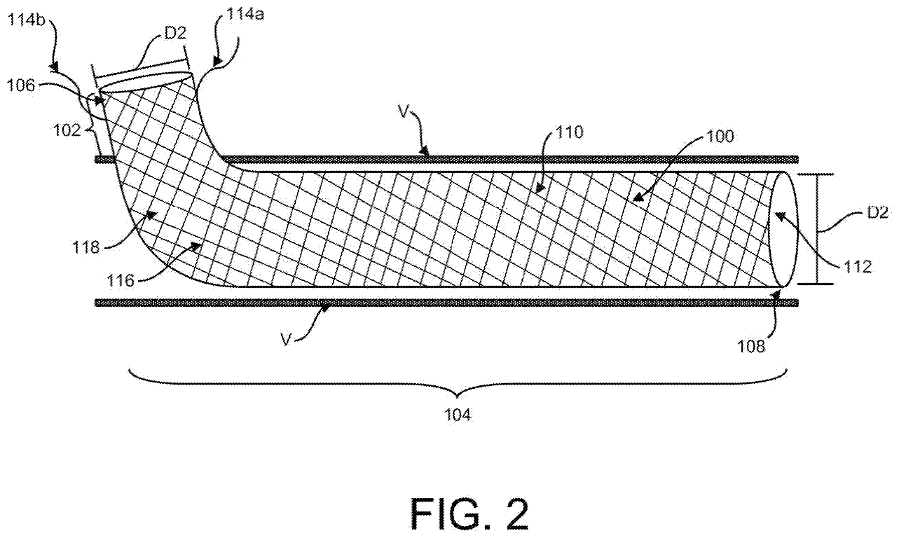

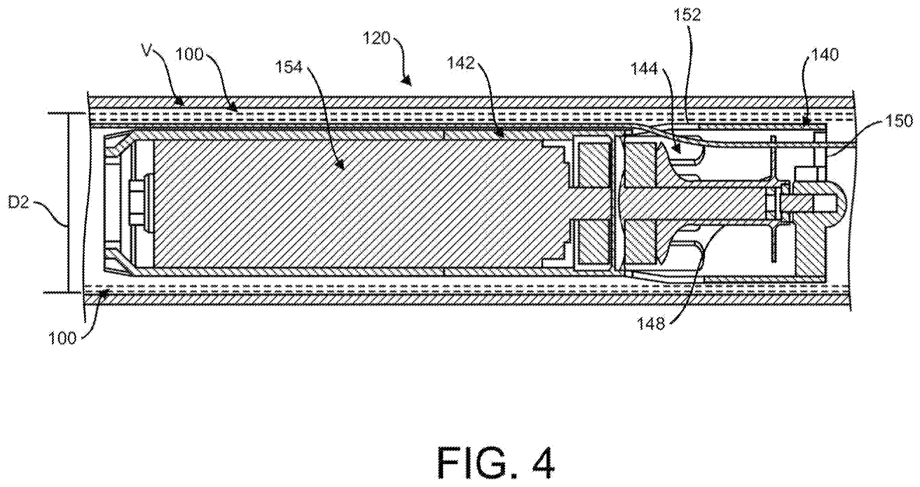

When the introducer sheath 100 is used for delivering a medical device, for example a percutaneous circulatory support device 130 (FIG. 4) (also referred to herein as the blood pump 130), the diameter of the introducer sheath 100 may need to be increased to accommodate passage of the blood pump 130, as will be described further with reference to FIG. 4. As such, the introducer sheath 100 may comprise at least one actuator for actuating at least the inner portion 104 of the introducer sheath 100 from the compressed configuration to the expanded configuration, as is illustrated in FIGS. 1 and 2. Specifically, FIG. 2 illustrates the introducer sheath 100 in the expanded configuration wherein the introducer sheath 100 is defined by an expanded diameter D2. The expanded diameter D2 has a value greater than the compressed diameter D1 and may be a pre-determined value based on the size that is required for allowing the delivery of a device, as will be described further with reference to FIG. 4. The second diameter D2 may defined by the introducer sheath 100 having a size of approximately 16 Fr. The value of the second diameter D2 may be varied and is not meant to be limited to the example as described above.

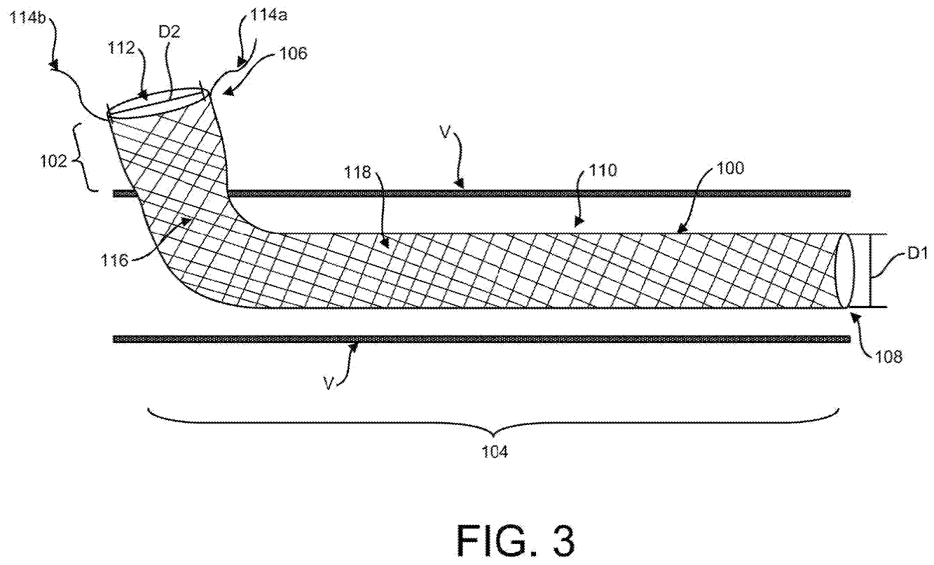

Additionally, as illustrated in FIGS. 1-2, both the inner portion 104 and the outer portion 102 are capable of being positioned in the expanded configuration and the compressed configuration. However, in various embodiments, only the inner portion 104 is configured for delivery and being positioned in the compressed configuration defined by the compressed diameter D1, while the outer portion 102 is configured in the expanded configuration defined by the expanded diameter D2 and does not compress to a compressed configuration. For example, this configuration is illustrated in the embodiment of FIG. 3, wherein the inner portion 104 is defined by the compressed diameter D1 while the outer portion 102 is defined by the expanded diameter D2. This may be advantageous to reduce the varying forces exerted against the blood vessel V at the incision site such that the blood vessel V is not potentially damaged by the changing of the diameter of outer portion 102 during insertion and/or removal of the medical device.

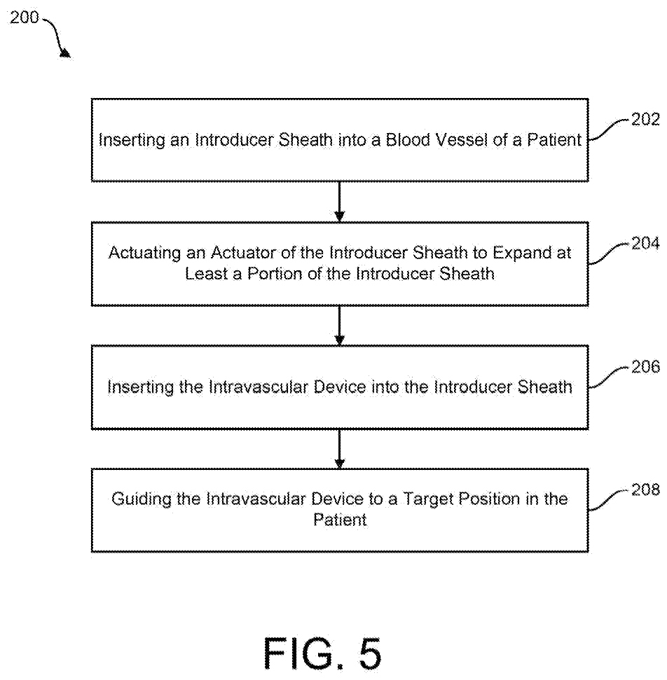

The actuation that allows this transition from the compressed configuration to the expanded configuration and vice versa will be described herein. Specifically, the actuator may include at least one pull wire. In the illustrative embodiment of FIGS. 1-3, the at least one pull wire includes a first pull wire 114a and a second pull wire 114b. However, in various other embodiments, the introducer sheath 100 may include more than two pull wires, for example, three, four, five or six pull wires. As illustrated, the first and second pull wires 114a, 114b are positioned opposite one another relative to the lumen 112 of the introducer sheath 100. However, the distribution of the pull wires 114a, 114b may be varied. Additionally, as previously mentioned, the introducer sheath 100 may be composed of the braided wire 116 and the pull wires 114a, 114b may be embedded within and extend from the braided wires 116 of the introducer sheath 100. The pull wires 114a, 114b may extend from the proximal end 106 of the introducer sheath 100 and extend proximally to a position wherein the physician or operator can actuate the pull wires 114a, 114b. Specifically, the pull wires 114a, 114b may be pulled by the operator in order to cause radial expansion of the introducer sheath 100. In this way, the plurality of pull wires 114a, 114b are configured for expanding at least the inner portion 104 from the compressed configuration (FIG. 1) to the expanded configuration (FIG. 2), prior to insertion of the percutaneous circulatory support device 130 into the introducer sheath 100. In other words, the physician may actively expand the diameter of at least the inner portion 104 so that the inner portion 104 can accommodate the passage of a medical device, as will be described further herein with reference to FIGS. 4 and 5.

FIG. 4 illustrates the percutaneous circulatory support device 130 positioned within the introducer sheath 100 when the introducer sheath 100 is defined by the second and expanded diameter D2. In this way, a medical device can be easily passed through the introducer sheath 100. Specifically, FIG. 4 illustrates an example of the introducer sheath 100 in use with a percutaneous circulatory support device 130, also referred to herein as a blood pump 130. As shown in FIG. 4, the introducer sheath 100 surrounds the blood pump 130 after both the introducer sheath 100 and blood pump 130 have been inserted at least partially within the blood vessel V. The blood pump 130 generally includes an impeller assembly housing 140 and a motor housing 142. In some embodiments, the impeller assembly housing 140 and the motor housing 142 may be integrally or monolithically constructed. In other embodiments, the impeller assembly housing 140 and the motor housing 142 may be separate components configured to be removably or permanently coupled. The impeller assembly housing 140 carries an impeller assembly 144 therein. The impeller assembly 144 includes an impeller shaft 146 and an impeller 148 that rotates relative to the impeller assembly housing 140 to drive blood through the blood pump 130. More specifically, the impeller 148 causes blood to flow from a blood inlet 150 formed on the impeller assembly housing 140, through the impeller assembly housing 140, and out of a blood outlet 152 formed on the impeller assembly housing 140. With continued reference to FIG. 4, the motor housing 142 carries a motor 154, and the motor 154 is configured to rotatably drive the impeller 148 relative to the impeller assembly housing 140. The above described blood pump 130 may pass through the entirety of the introducer sheath 100 and out of the distal end 108 to be delivered to a target location in the patient such as the left ventricle of the heart.

As will be described further with reference to FIG. 5, once the delivery of the percutaneous circulatory support device 130 is complete, the introducer sheath 100 may be collapsed back down into the compressed diameter D1 using the aforementioned pull wires or similar components. While described above with reference to the percutaneous circulatory support device 130, the introducer sheath 100 as described herein may be used with any variety of relatively large bore medical devices for delivering the medical devices to a target location within a patient.

FIG. 5 illustrates a flow chart of the method of delivering a medical device, for example the percutaneous circulatory support device 130 as described with reference to FIG. 4, through the introducer sheath 100 and to a target location in the patient.

At block 202, the method 200 includes inserting the introducer sheath 100 into the blood vessel V of the patient. During this step, the introducer sheath 100 is positioned such that the inner portion 104 of the introducer sheath 100 extends within the blood vessel V while the outer portion 102 of the introducer sheath 100 is positioned outside of the blood vessel V.

At block 204, the method 200 includes actuating an actuator of the introducer sheath 100 to expand at least a portion of the introducer sheath 100. Specifically, for example, the pull wires 114a, 114b may be actuated such that at least the inner portion 104 expands to the expanded configuration having the expanded diameter D2. In further embodiments, the actuation of the pull wires 114a, 114b may cause the expansion of both the inner portion 104 and the outer portion 102 of the introducer sheath 100.

Further, at block 206, the method 200 further includes inserting the percutaneous circulatory support device 130, into the introducer sheath 100. As the pull wires 114a, 114b were previously actuated to the expanded configuration, the percutaneous circulatory support device 130 is able to readily fit within the introducer sheath 100 and be pushed through the introducer sheath 100 from the proximal end 106 to the distal end 108. The method 200 then includes the step at block 208 which comprises guiding the introducer sheath 100 to a target position in the patient. Once the percutaneous circulatory support device 130 is positioned outside of the introducer sheath 100, the tension in the pull wires 114a, 114b may be released such that the introducer sheath 100 can be compressed back down to the compressed configuration. In this way, while the introducer sheath 100 is not needed for receiving the device 130, the introducer sheath 100 does not impart a radial force against the blood vessel V. In this way, the amount of force over the time of the procedure is reduced, which may reduce damage against the blood vessel V, the introducer sheath 100, and/or the percutaneous circulatory support device 130. In addition, the introducer sheath 100 takes up less space in the blood vessel V in the compressed configuration, allowing for blood flow around the introducer sheath 100 and through the blood vessel V.

In various embodiments, prior to the removal of the percutaneous circulatory support device 130, the pull wires 114a, 114b may be actuated to expand the introducer sheath 100 once again to the expanded configuration. In this way, the introducer sheath 100 is expanded to accommodate the percutaneous circulatory support device 130 as it is removed from the target location in the patient and through the introducer sheath 100 and out of the proximal end 106 of the introducer sheath 100.

The ability of the introducer sheath 100 to be actively expanded through the actuation of the pull wires 114a, 114b in contrast to a passive expansion of the introducer sheath 100 based on the device being pushed into the introducer sheath 100, results in a lower force value being required for expanding the introducer sheath 100 applied to the blood vessel V in comparison with a passively expanded introducer sheath. In other words, the amount of force imparted to the blood vessel V caused by the expansion of the introducer sheath 100 is less than the amount of force that would be required to push the percutaneous circulatory support device 130 through the introducer sheath that relies on passive expansion of the introducer sheath.

One further advantage of the introducer sheath 100 as described herein is that it may eliminate the need for a separate repositioning sheath to be incorporated into the system. That is, the introducer sheath 100 may remain in place the entire time that the medical device is in the patient as the introducer sheath 100 does not need to be replaced with a smaller sheath that occupies less space in the blood vessel V and thus allows for improved blood flow through blood vessel V. Avoiding removal of the introducer sheath 100 and the insertion of an additional sheath may reduce the instances of traumatic injury to the blood vessel V and may reduce bleeding or other adverse effects at the incision site.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above-described features.

1. An introducer sheath for use in delivering a percutaneous device, the introducer sheath comprising:
a proximal end opposite a distal end and a body portion extending between the proximal portion and the distal portion, the body portion defining a lumen of the introducer sheath configured for receiving at least one percutaneous device;
a first portion of the introducer sheath having a compressed configuration defined by a compressed diameter and an expanded configuration defined by an expanded diameter, the compressed diameter being less than the expanded diameter;
at least one actuator that is configured for transitioning the first portion from the compressed diameter to the expanded diameter and configured for actuation prior to insertion of the percutaneous device through the introducer sheath.

2. The introducer sheath of claim 1, wherein the actuator includes at least one pull wire.

3. The introducer sheath of claim 1, wherein at least the first portion of the introducer sheath is composed of a braided wire.

4. The introducer sheath of claim 3, wherein the at least one pull wire is an extension of the braided wire of the first portion.

5. The introducer sheath of claim 1, wherein the first portion extends along an entire length of the introducer sheath.

6. The introducer sheath of claim 1, wherein the first portion extends from the distal end of the body portion and extends a length less than an entire length of the introducer sheath.

7. The introducer sheath of claim 6, wherein the introducer sheath comprises a second portion that extends from the proximal end and extends a length less than the entire length of the introducer sheath, and wherein the second portion is defined by the expanded configuration and does not compress to the compressed configuration.

8. The introducer sheath of claim 6, wherein when in the compressed configuration, the first portion is defined by a size of 9 French.

9. The introducer sheath of claim 1, wherein when in the expanded configuration, the first portion is defined by a size of 16 French.

10. A percutaneous device delivery system for delivering a percutaneous device, the system comprising:
a percutaneous device comprising
an impeller housing for supporting an impeller,
a motor configured to rotatably drive the impeller within the impeller housing; and
an introducer sheath for receiving the percutaneous device, the introducer sheath having a proximal end, a distal end, and a body portion extending between the proximal portion and the distal portion, the body portion defining a lumen, the introducer sheath further including:
a first portion extending from the distal end of the introducer sheath, the first portion having a compressed configuration defined by a compressed diameter and an expanded configuration defined by an expanded diameter,
an actuator configured for transitioning the first portion from the compressed configuration to the expanded configuration after actuation.

11. The delivery system of claim 10, wherein the actuator includes a plurality of pull wires extending from the proximal end of the introducer sheath.

12. The delivery system of claim 10, wherein at least the first portion is composed of a braided wire.

13. The delivery system of claim 10, wherein the introducer sheath comprises a second portion extending from the proximal end, and wherein the second portion is defined by the expanded configuration.

14. The delivery system of claim 13, wherein the second portion does not compress to the compressed configuration.

15. A method for delivering a percutaneous device into a patient; comprising:
inserting an introducer sheath into a vessel of the patient, the introducer sheath comprising a proximal end opposite a distal end, a body portion extending between the proximal end and the distal end and the body portion defining a lumen, and the introducer sheath having a first portion extending from the distal end wherein the first portion has a compressed configuration defined by a compressed diameter and an expanded configuration defined by an expanded diameter, the first portion having the compressed diameter when the inserting the introducer sheath;
actuating an actuator of the introducer sheath such that the first portion expands from the compressed diameter to the expanded diameter;
inserting the percutaneous device into the introducer sheath; and
guiding the percutaneous device to a target position in the patient.

16. The method of claim 15, wherein the actuator includes at least two pull wires extending from the proximal end of the introducer sheath.

17. The method of claim 16, wherein the introducer sheath is composed of a braided wire and wherein the at least two pull wires extend from the braided wire.

18. The method of claim 15, wherein the introducer sheath comprises a second portion extending from the proximal portion having the expanded configuration defined by the expanded diameter.

19. The method of claim 18, wherein the second portion does not compress to the compressed configuration.

20. The method of claim 16, the method further including the step of compressing the first portion from the expanded configuration to the compressed configuration through releasing tension of the at least two pull wires.
